(19) United States
(12) Plant Patent Application Publication
(10) Pub. No.: US 2018/0213691 P1
(43) Pub. Date: Jul. 26, 2018

(54) Prunus rootstock 'WRM-2'

(50) ;

(71) Applicant: James F. Doyle, Roseville, CA (US)

(72) Inventor: James F. Doyle, Roseville, CA (US)

(21) Appl. No.: 15/530,511

(22) Filed: Jan. 23, 2017

(51) Int. Cl. A01H 6/74

(52) U.S. Cl. PLT/180

(57) ABSTRACT

A new and novel Prunus Rootstock is described and which has a vigorous growth habit, and which further shows a high level of tolerance to being grown in a high moisture environment.

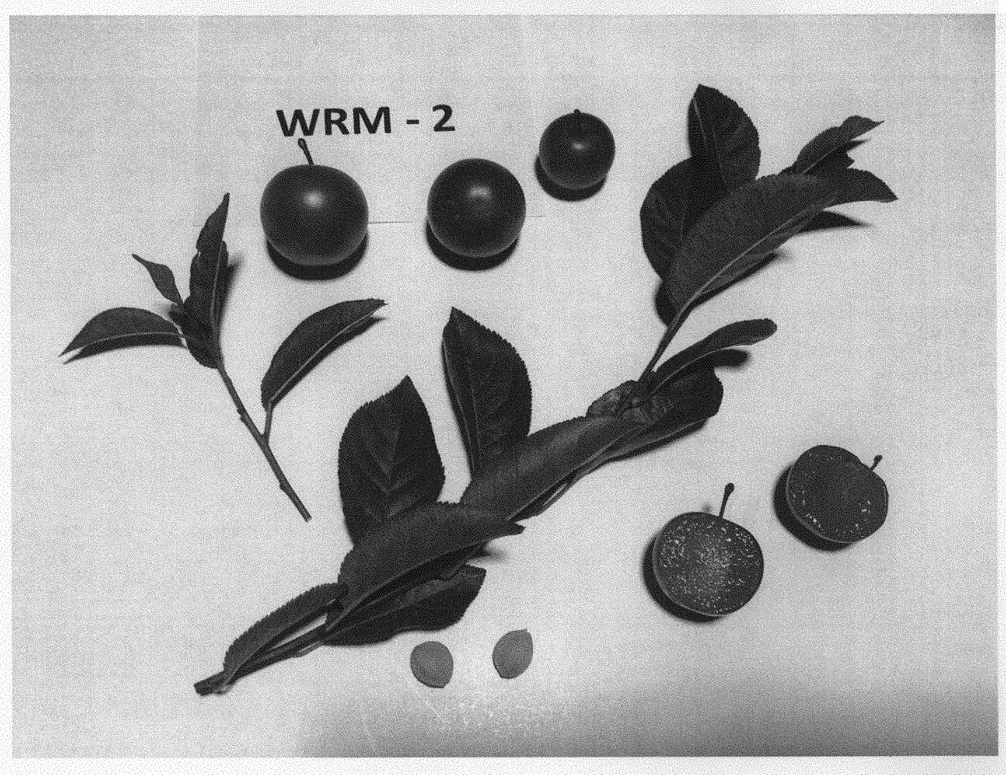

Latin name: Prunus cerasifera.

Varietal denomination: WRM-2.

### BACKGROUND OF THE NEW VARIETY

The present invention relates to a new novel and distinct variety of rootstock for use in growing prune trees (Prunus domestica), and other commercially valuable fruit tree varieties, and which further has been denominated varietally as ‘WRM-2’. The present variety of rootstock was developed from seed collected in the summer of 2005 from a red-leafed myrobalan-type plum tree (Prunus cerasifera), and which was growing within a cultivated region along, and on, the bank of the Willamette River which is located near Springfield, Oreg. The originally discovered tree, which was found by the inventor growing within this cultivated area, had been growing in this very wet environment on this river bank for a number of years inasmuch as the newly discovered tree had a tree trunk which was approximately 10 inches in diameter. At the time of the discovery, the trunk of the originally discovered tree was nearly surrounded by moving water. The inventor collected 10 to 12 seeds from the newly discovered tree, and subsequently developed, and tested the new seedlings from the seeds that were so collected. In view of the demonstrated tolerance the newly discovered tree had to these extremely wet soil conditions, the inventor reasoned that a new rootstock could perhaps be developed for use with plum and other fruit trees which are exposed to similar wet soil conditions.

The seeds removed from the originally discovered parent tree were taken to the inventor's residence in Pleasant Hill, Oreg., where the seed was dried, cleaned and stored for future planting. In 2006 the inventor moved from Pleasant Hill, Oreg., to Chico, Calif. During the 2006 growing season the aforementioned collected seed was stratified, germinated and planted in small pots which were located at the inventor's residence. An irrigation system was then developed by the inventor for these pots and which kept the soil in the pots at a constantly high level of moisture. The objective of this type of irrigation was to challenge the ability of each seedling in this small population of seeds to germinate, grow and survive under high soil moisture conditions similar to the demonstrated high moisture tolerance displayed by the originally discovered parent tree. After several months of growth under these wet conditions most of the seedlings had either died, or were growing poorly. One seedling, however, germinated, and began to grow vigorously, and displayed no readily apparent adverse effects from the very high moisture regimen which had been provided. This vigorous seedling, which displayed red leaves, especially on immature shoots, and near the terminals, was later named ‘WRM-2’. The remaining surviving seedlings that had performed less favorably, were then discarded.

In 2007 the inventor moved from Chico, Calif. to Roseville, Calif. The newly-discovered tree, and which has been designated ‘WRM-2’ was then planted from its original container into a palmette-style row of experimental trees at Roseville, Calif., where it remains today. In 2008, the new tree was top worked, by grafting, to a virus free Japanese plum (Prunus salicina). This variety is commonly referred to as ‘Elephant Heart’ (unpatented). This grafted tree has developed into a large tree, and continues to grow and produce fruit at the present geographic location.

Hardwood cuttings of the newly discovered rootstock ‘WRM-2’ were made by the inventor in 2008. These hardwood cuttings were rooted successfully, by asexual propagation. Many hardwood cuttings of the newly discovered variety have been rooted satisfactorily since the original 2008 cuttings, and have displayed the same unique and novel characteristics of the newly discovered tree. The bulk of these cuttings have been budded to the improved ‘French’ prune variety (Prunus domestica), (unpatented), and have been established in experimental field tests. Subsequent compatibility testing of the new plant variety ‘WRM-2’ to the aforementioned ‘French’ prune, and the growth of the improved ‘French’ prune top on ‘WRM-2’ is excellent. In the oldest experimental field trial which was planted in 2011, and in comparison with numerous other well-known rootstocks for use with prunes, including the commercial standards, the new rootstock ‘WRM-2’ has so far demonstrated superior tree size, and productivity.

### SUMMARY OF THE NEW VARIETY

The new rootstock, ‘WRM-2’ (Prunus cersifera) is considered to be a myrobalan plum tree. Generally speaking Myrobalan plum trees (unpatented) have been used successfully as a rootstock in the prune industry for many years. The most frequently used variety of myrobalan plum tree employed as a rootstock is the ‘Myro 29C’ (unpatented). In comparison with the aforementioned commercial variety of rootstock the new variety ‘WRM-2’ is clearly distinguishable inasmuch as the new variety produces red leaves especially on immature wood that is near the shoot terminals. These red leaves provide increased benefit when the new rootstock is used in a nursery row. For example, when a prune tree is propagated on ‘Myro 29C’ rootstock, the coloration of the green colored prune top closely resembles the green color of the leaves produced by the ‘Myro 29C’ rootstock. If the budding or grafting of the new prune top is unsuccessful less skilled nursery workers in the field can mistake the growth of the leaves of the ‘Myro 29C’ rootstock for the growth of the new propagated top which may be, for example, from a ‘French’ plum (unpatented). This can result in nursery workers not removing unsuccessful propagations from a nursery row. The result is that unsuccessful propagations may become mixed in with successfully propagated trees. As should be understood, the presence of the red leaves as produced on the shoot terminals of the ‘WRM-2’ rootstock simplifies the correct identification of rootstock growth, as compared to a successful propagation, for example of a ‘French’ plum onto the same rootstock. No other ‘Myrobalan’ type prune rootstock, which is currently available, produces this type of red leaves. Further the ‘WRM-2’ Prunus rootstock appears to have a high level of tolerance to wet growing conditions. Since prunes may on occasion be grown on what is considered heavier and wetter-type soils, a reasonable expectation exists that prune trees propagated on ‘WRM-2’ rootstock will perform better than less adapted rootstocks when grown under similar, challenging, wet soil conditions. In addition to the foregoing, the new rootstock has demonstrated a high level of vigor resulting in larger tree sizes, and has also demonstrated increased crop yields due to the larger sized volume of the tree. It should be understood that much variability of tree size, and yield can occur within a range of soil types, and fertility in orchard locations so there may be a possibility of substantial variability in tree performance from location-to-location. The present variety has shown a high compatibility when budded with a ‘French’ prune (Prunus domestica). Ongoing testing is taking place to determine the usefulness of the present rootstock with other species and varieties other than ‘French’ prunes.

### BRIEF SUMMARY OF THE DRAWING

The accompanying drawing which is provided is a color photograph of several parts of the new rootstock variety. The photograph depicts two whole mature fruit which have been severed in the longitudinal plane; three mature fruit showing the dorsal, ventral and exterior characteristics thereof; a sample vegetative shoot showing the leaf characteristics thereof; and a sample of a vegetative shoot terminal and which displays a characteristic red leaf coloration. The colors in this photograph are as nearly true as is reasonably possible in a color representation of this type. Due to chemical development, processing, and printing the leaves and fruit depicted in this photograph may, or may not be accurate when compared to the actual specimen. For this reason, future color references should be made to the color designations as found in The Royal Horticultural Society Colour Chart, 3^{rd} Edition and which was published in 1995.

### NOT A COMMERCIAL WARRANTY

The following detailed description has been prepared to solely comply with the provisions of 35 U.S.C. § 112, and does not constitute a commercial warranty, (either express or implied) that the present variety will, in the future, display all of the botanical, pomological or other characteristics as set forth hereinafter. Therefore, this disclosure may not be relied upon to support any future legal claims including, but not limited to, breach of warranty of merchantability, or fitness for any particular purpose, or non-infringement which is directed, in whole, or in part, to the present rootstock variety.

### DETAILED DESCRIPTION

Referring more specifically to the pomological details of this new and distinct variety of rootstock, the following has been observed during the fourth growing season and under the ecological conditions prevailing at the orchard of the inventor and which is located near Roseville, Calif. All major color code designations are by reference to the R.H.S. Colour Chart published by The Royal Horticultural Society of Great Britain, 3rd Edition 1995. Common color names are also occasionally used.
Tree:
   Tree vigor.—Considered vigorous for the species.
   Tree growth.—Considered upright to upright spreading in form. The tree that is described was trained into a two-leader palmette form.
   Tree productivity.—Considered productive. The new rootstock has produced regular crops of small cherry-like fruit. The fruit produced by the new variety ‘WRM-2’ is not commercially important. The present value of the new plant is that it is useful from the standpoint of being a commercial rootstock.
   Hardiness.—The new rootstock is considered hardy under typical environmental and climatic conditions as experienced in the Sacramento Valley of Northern California.
   Tree height.—About 3.5 to 3.8 meters.
   Tree width.—Approximately 2.2 meters.
   The amount of the current seasons growth.—On average about 1 to about 1.5 meters.
Trunk:
   Trunk diameter.—When measured on a four year old tree, the diameter is about 6 cm when measured at a point which is about 10 cm from the surface of the earth.
   Bark texture.—Considered rough, and having longitudinal striations, and some low scarfskin is evident.
   Bark color.—Variable, from a brown color, (Fan 4, Sheet 177A), to a lighter brown color, (Fan 4, Sheet 177B).
   Bark lenticels.—Numbers — Numerous.
   Bark lenticels.—Shape — Generally oval in shape; about 3 to about 4 mm in length; and further having a height dimension of about 1 to 2 mm. The lenticels are flattened in the horizontal plane.
   Lenticel surface texture.—Calloused and having a light brown color (Fan 4, Sheet 164A).
   Scaffold branches.—Diameter — About 3.4 to about 4.1 cm when measured at a location where the scaffold branches arise from the trunk.
   Scaffold branch bark texture and color.—These characteristics are substantially identical to that of the trunk.
   Secondary branches.—Thickness — Variable from about 18 to 19 mm in diameter for the larger secondary branches; and about 5 to about 13 mm, and sometimes smaller in diameter, for the smaller secondary branches.
   Secondary branch color.—Similar to that of the scaffold branches and ranging from a medium brown color (Fan 4, Sheet 177A), to a lighter brown color (Fan 4, Sheet 177B).
   Secondary branch scarfskin.—Present, and appearing moderately striated and further having a gray color (Fan 4, Sheet 197A).
   Secondary branch surface texture.—Generally — Glabrous for mature secondary branches.
   Immature secondary shoots.—Color — Generally speaking a transition in color occurs as Immature secondary shoots mature. This change in color progresses from a dark purple-red color (Fan 4, Sheet 187B) when immature, to a dark brown color (Fan 4, Sheet 200C) when fully mature.
   Internode length.—The internode length on current season's shoots is variable but within the range which is normally expected. The internode length can vary from about 1.5 to about 5 cm on small, to medium length shoots; and from about 3 to about 10 cm on larger, and more rapidly expanding shoots.
   Expanding shoot tips.—Color — Reddish-purple (Fan 4, Sheet 181B).
Leaves:
   Generally.—The leaves of the new variety of rootstock are considered medium in size, and average in thickness. The measurements which follow were taken from leaves growing at, or near mid-shoot, on vigorous, upright, current season's shoots.
   Leaf length.—About 7 to about 8.9 cm, not including the leaf petiole.
   Leaf width.—Variable, from about 3.9 to 5.1 cm.
   Leaf thickness.—Average for the species.
   Leaf surface texture.—Generally speaking it is glabrous, although occasionally, the surface texture of the lower leaf veins may display a light pubescence.
   Leaf form.—Generally — Oval.
   Leaf tip.—Form — Acuminate. The tip often appears slightly twisted in a sideways (lateral) direction.
   Leaf mid vein.—Generally speaking the mid-vein is considered prominent especially when viewed from the lower leaf surface.
   Mid-vein color.—Variable, light yellow in color, (Fan 3, Sheet 148D), to (Fan 3, Sheet 151C).
   Mid-vein thickness.—About 1 to about 1.5 mm when measured at its widest point.
   Leaf color.—Generally — The leaf color as found on the new variety of rootstock ‘WRM-2’ can vary substantially as the tree matures.
   Upper leaf surface color.—Immature leaves can vary from a medium-red color, (Fan 4, Sheet 180C), to a darker red color, (Fan 4, Sheet 181B).
   Lower leaf surface color.—Immature leaves can vary from a medium red, (Fan 4, Sheet 180B), to a darker red color, (Fan 4, Sheet 181B). Generally speaking as the leaves mature, there is a gradient in color development, from red to green, with many shades and variations, therebetween.
   Upper leaf surface color.—Mid Season: Leaves in the mid-season of maturity have a dark green color, (Fan 3, Sheet 147A). The lower leaf surfaces, of mid-season leaves, display a slightly lighter green color, (Fan 3, Sheet 147B to Sheet 147C). However, it should be understood that even the fully expanded, and mature leaves can retain some light shading of reddish coloration primarily along the leaf margins and near the primary central leaf vein.
   Leaf marginal form.—Considered crenate and occasionally double crenations are observed.
   Crenation size.—Average. Occasionally some serrations may be present along the marginal edge.
   Marginal undulation.—A small amount of undulation is observed along the leaf margin.
   Leaf petiole.—Average length — About 12 to 17 mm.
   Leaf petiole.—Thickness — about 1.2 to about 1.5 mm.
   Leaf petiole color.—Light green, (Fan 3, Sheet 145B) to a more yellow-green, (Fan 3, Sheet 154C). The petiole is often overlain with a pale, purplish blush which extends basally, and along the ridges subtending the petiole groove, (Fan 4, Sheet 182D).
   Leaf petiole surface texture.—Glabrous, except along the top of the petiole ridges where a light pubescence may be observed.
   Leaf glands.—Size — Considered very small, and less than 0.5 mm in diameter, when observed.
   Leaf glands.—Shape — When present, the leaf glands are globose, or bulbous in form, and further located along the tip of the basal leaf serrations.
   Leaf gland.—Color — Considered a medium-red color, (Fan 4, Sheet 78D). The gland color darkens with advancing senescence.
   Leaf stipules.—Length — About 4.0 to 7.0 mm.
   Leaf stipules.—Width — About 1 mm, when measured basally.
   Leaf stipules.—Numbers — Usually 2 per node are found. The leaf stipules are lanceolate in form, but often split in two parts near the base.
   Leaf stipule margins.—Shape — Serrate. The serrations are widely spaced, and the tip can be somewhat bulbous in appearance.
   Leaf stipule color.—Red when viewed basally, (Fan 4, Sheet 181D), but having a more greenish color when viewed near the stipule tip, (Fan 3, Sheet 153C). The stipules are considered early deciduous.
Fruit: The following fruit description, and other botanical observations were taken from fruit samples collected from trees growing in the orchard of the inventor, and which is located near Roseville, Calif. The color definitions, as used hereinafter, were taken from The Royal Horticulture Society Colour Chart, 3^{rd} Edition, and which was published in 1995.
   Fruit maturity.—Date of first pick, May 25, 2015 at Roseville, Calif.
   Fruit maturity.—Date of Last Pick, Jun. 5, 2015 at Roseville, Calif.
   Fruit size.—Generally — Uniformly small.
   Fruit cheek diameter.—About 26 mm to 32 mm.
   Suture diameter.—About 26 to about 32 mm.
   Axial diameter.—About 24 mm to 28 mm.
   Fruit form.—Generally — Symmetrical, and most frequently globose when viewed in both the lateral, and apical aspects.
   Fruit suture.—Generally — The fruit suture appears as a thin, inconspicuous line which extends from the apex, to the base of the fruit. There is no apparent clefting or depression observed along the suture line.
   Suture color.—Red (Fan 4, Sheet 179A). Occasionally some striping appears in a location which is parallel to the suture line. This parallel striping may have a variable, red coloration ranging from (Fan 4, Sheet 185A), to (Fan 4, Sheet 181A).
   Fruit ventral surface.—Surface Texture — Considered smooth, with no lipping or callous being present.
   Fruit stem cavity.—Size — Small, round, and regular in form.
   Stem cavity width.—About 5 to 8 mm.
   Stem cavity length.—About 5 to 8 mm.
   Stem cavity depth.—About 1 to 2 mm.
   Fruit base.—Generally — Considered round, and slightly truncate in form. The base is usually oriented at right angles to the fruit axis.
   Fruit apex.—Generally — Considered round, and occasionally having a small dimple which is located right at the pistil point. The pistil point is in the apical position.
   Fruit stem.—Length — About 13 to 16 mm.
   Fruit stem.—Width — About 1 to 2 mm.
   Fruit stem color.—Purple at full maturity, (Fan 4, Sheet 187A).
   Fruit stem surface texture.—Glabrous.
   Fruit skin generally.—Considered relatively thin, and fully attached to the fruit flesh.
   Fruit skin surface texture.—Glabrous.
   Fruit skin flavor.—Considered highly acidic.
   Tendency to crack.—Not observed.
   Fruit skin color.—Generally — The fruit skin is usually 100% colored and has a moderately uniform red-purple coloration, (Fan 4, Sheet 180A) and which varies to a darker-red violet when fully mature, (Fan 4, Sheet 184A). Some light speckling may be observed at full maturity. The speckling appears associated with the fruit lenticels, and can range in color from a light red, (Fan 2, Sheet 66C), to a darker red, (Fan 4, Sheet 185D).
   Fruit flesh color.—Considered dark red in areas located just under the skin, (Fan 4, Sheet 179A). The flesh located near the stone has a lighter color of red, (Fan 4, Sheet 169B).
   Fruit flesh texture.—Considered soft, fibrous and juicy.
   Fruit ripening generally.—The fruit ripens evenly.
   Fruit flavor and aroma.—The flesh located away from the stone and the juice are sweet and pleasant. On the other hand the fruit skin, and the flesh near the stone, are very acidic. Fruit aroma is generally lacking.
   Fruit eating quality.—Considered uniformly poor. The fruit is overall too acidic and cannot be recommended for consumer consumption.
Stone:
   Generally.—Clingstone and having fibers attached over most of the stone surface, and which further radiate outwardly towards the fruit skin.
   Stone size.—Generally — Considered small, about 13 to about 15 mm in length; and about 10.5 to 13.5 mm in width. The stone has a thickness of about 7 to about 9 mm.
   Stone form.—Generally — Oval.
   Stone base.—Generally — Considered slightly truncated. Still further the base angle is variable, and is most frequently at a right angle to the stone axis. Occasionally a very slightly oblique stone axis may be observed.
   Stone hilum.—Generally — Small in size, and oval in form.
   Stone hilum.—Surface Texture — The area around the stone hilum is moderately eroded.
   Stone apex.—Shape — Rounded, and having almost no tip present.
   Stone sides.—Shape — Generally considered equal.
   Stone surface texture.—Generally — Relatively smooth laterally, and having slight dimpling over the apical shoulders near the dorsal ridge. Further, two grooves are present. One groove appears on each side of the ventral ridge. These grooves are roughly parallel to the ventral edge, and converge with the ventral edge both apically, and basally.
   Stone ventral edge.—Width — About 3 to 4 mm when measured at mid-suture.
   Ventral edge wings.—Form — The wings are low, relatively smooth, and converge with the ventral edge ridge both apically, and basally. The wings are relatively uniform in shape, and vary from 1 to about 2 mm at their maximum height.
   Stone dorsal edge.—Generally — A relatively wide groove extends from the stone base, over the basal shoulder, and to about mid-stone. This basal shoulder groove varies from about 1 to about 2 mm in width. Over the apical shoulder the dorsal edge groove is much thinner, and appears only as a thin line.
   Stone color.—Generally — Variable from a medium tan color, (Fan 4, Sheet 165C), to a lighter tan, (Fan 4, Sheet 165D). A light amount of purple staining can be observed on the surface of the stone, and especially along the stone suture areas, (Fan 4, Sheet 181D).
   Tendency to split.—There is no observed tendency for this stone to split.
Flowers: The flowers were observed from specimens that were collected on Mar. 2, 2015 at Roseville, Calif.
   Date of first bloom.—Feb. 19, 2015 at Roseville, Calif.
   Date of full bloom.—Feb. 28, 2015 at Roseville, Calif. This time of bloom, roughly coincides with the full bloom of Japanese plum trees, (Prunus salicina), (unpatented), and which have similar chilling requirements, and are further growing in approximately the same geographical location.
   Flower size.—Generally — Small to medium in size.
   Flower diameter.—When fully opened about 1.7 to 2.5 cm. The overall flower size can vary somewhat depending on the total volume of bloom on the observed tree.
   Number of petals.—5. Occasionally a flower may be found with 6 petals.
   Bloom quantity.—Considered abundant for the species.
   Flowers per node.—Variable from 1 to 4 flowers are observed. Typically 2 flowers are present per node.
   Petal size.—Length — Variable from 9.5 to 10.5 mm, including the petal claw.
   Petal width.—about 7.5 to 8.5 mm when measured across the widest area of the petal.
   Petal claw.—Shape — Short and truncate in form.
   Petal claw.—Width — About 1 to 1.5 mm.
   Petal claw.—Length — Variable, from about 1 to about 1.5 mm.
   Petal form.—Most frequently ovate, and having a slightly to moderately undulate petal margin. The individual petals cup slightly, upwardly, when the petals are fully expanded.
   Petal color.—Considered a very light pink, (Fan 1, Sheet 56D). This color fades to an even lighter pink, and almost white color, with increasing senescence.
   Petal claw.—Color — The petal claw is a darker pink at the base, and varies from a pink color, (Fan 2, Sheet 62B to Sheet 63C).
   Pedicel size.—Variable from about 6 mm to as long as 13.5 mm.
   Pedicel thickness.—about 1 mm.
   Pedicel color.—Reddish-violet, (Fan 4, Sheet 183C).
   Calyx surface texture.—Generally smooth on the outer surface. The calyx surface texture is essentially glabrous.
   Calyx diameter.—Variable from about 3 to about 4 mm at the widest point.
   Calyx outer surface color.—Reddish-purple, (Fan 4, Sheet 182B). Regular dark red vertical stripes are usually present, (Fan 4, Sheet 183C).
   Sepals.—Numbers — 5.
   Sepals.—Form — Conic and highly reflexed.
   Sepals.—Upper and Lower Surface Textures — Glabrous.
   Sepal margin.—Shape — Serrate.
   Sepal length.—About 2.5 to 3 mm. This measurement was taken at full expansion.
   Sepal width.—About 2 to about 3.5 mm when measured at the widest point.
   Sepal color.—Green in the central area, (Fan 3, Sheet 144B), and having a red-violet colored margin, (Fan 2, Sheet 61C).
   Floral nectaries.—Color — Brown, (Fan 4, Sheet 175C).
   Anther size.—About 1 mm in length, and about 0.7 mm in width.
   Anther shape.—Plump.
   Anther color.—The dorsal side of each anther is yellow in color, (Fan 1, Sheet 17D), and further has a light reddish colored margin, (Fan 1, Sheet 37A). The ventral side of each anther has a similar color combination.
   Filament.—Length — About 6.0 to 9.0 mm.
   Filament color.—Variable, from white to light pink, (Fan 1, Sheet 56D) on the upper part of the filament, and a deeper pink, basally, (Fan 2, Sheet 62D).
   Pollen amount.—Considered abundant.
   Pollen color.—Yellow-gold, (Fan 1, Sheet 13B).
   Pistil.—Length — About 7 to about 10 mm, including the length of the ovary.
   Pistol surface texture.—Glabrous.
   Pistol color.—Purple, (Fan 2, Sheet 60D).
   Ovary.—Size — About 1 to 2 mm in length; and about 0.5 to 1 mm in width.
   Ovary surface texture.—Glabrous.
   Ovary color.—Principally purple, (Fan 2, Sheet 60C), but occasionally the ovary can have some greenish shading especially basally.
   Dormant flower bud description.—Generally — The following information was developed from dormant buds that were collected at Roseville, Calif. on Jan. 26, 2015.
   Bud size.—Generally — Small and ranging in length from 1.5 to 2 mm; and about 1 mm in width when measured at the bud base.
   Bud form.—Generally conic, slightly elongated, and relatively plump.
   Bud site character.—The dormant flower buds are relatively free from the underlying stem.
   Bud count per node.—Generally — The flower bud count can vary between nodes, from 0 to 4, and occasionally more. Typically 1 to 2 floral buds are observed.
   Bud surface characteristics.—The dormant flower bud surface is generally glabrous. The flower bud surface color is generally a dark brown, (Fan 4, Sheet 200C).
   Dormant bud size.—Generally — Dormant shoot buds are small in size. The bud length varies from about 1 mm to about 1.5 mm, and the bud width also varies from about 1 mm to about 1.5 mm, when measured at the bud base.
   Dormant bud form.—The leaf buds are conic in form, and somewhat broader, but less plump than the floral dormant buds.
   Dormant bud site character.—The dormant leaf buds are moderately appressed relative to the bearing shoot.
   Bud count, per node.—Typically there is only one leaf bud per node.
   Bud surface character and color.—The surface of the leaf bud is generally glabrous. Further, the surface of the leaf bud is brownish-purple in color, (Fan 4, Sheet 183A).
   Leaf bud hardiness.—The leaf buds of the present variety of plant are considered hardy under the typical climatic, and environmental conditions which are experienced in the Sacramento Valley of Northern California.
   Resistance to insects and disease.—No particular susceptibilities were noted. The present variety has not been intentionally tested to expose or detect any susceptibilities or resistances to any known plant or fruit diseases, insect, frost, winter injury or other ambient environmental factors. Although the new variety of rootstock possesses the described characteristics when grown under the ecological conditions prevailing in the Sacramento Valley of Northern California, it should be understood that variations of the usual magnitude and characteristics incident to changes in growing conditions, fertilization, nutrition, pruning, pest control, frost, climatic variables and changes in horticultural management and other practices are to be expected.

Having thus described and illustrated my new variety of

Prunus

rootstock, what I claim is new, and desire to secure by plant Letters Patent is:

1. A new and distinct variety of Prunus Rootstock as substantially illustrated and described and which is principally characterized as to novelty by displaying a vigorous growth habit, and which further shows a high level of tolerance to being grown in a high moisture environment.
